(19) United States
(12) Patent Application Publication
(10) Pub. No.: US 2012/0022621 A1
(43) Pub. Date: Jan. 26, 2012

(54) THERMAL DEVICE

(75) Inventors: Vincent York-Leung Wong, Hamilton, OH (US); Mary Elaine Freeland, Loveland, OH (US); James Patrick Ebel, Lebanon, OH (US)

(73) Assignee: Wyeth LLC, Madison, NJ (US)

(21) Appl. No.: 13/197,874

(22) Filed: Aug. 4, 2011

Related U.S. Application Data
(63) Continuation-in-part of application No. 12/052,296, filed on Mar. 20, 2008.
(60) Provisional application No. 60/919,008, filed on Mar. 20, 2007.

(51) Int. Cl. A61F 7/08

(52) U.S. Cl. 607/96

(57) ABSTRACT

A method of delivering safe therapeutic heat to an unconscious or inert subject is described, wherein a disposable device is applied to the user that provides a consistent skin side temperature, wherein said device comprises a thermal source comprising a particulate exothermic composition; and a primary insulative material disposed on a skin side of the thermal source that delivers heat while protecting the subject from burns; wherein said device provides a rate of temperature change of less than about 0.8.

### CROSS REFERENCE TO RELATED APPLICATION

This application is a continuation-in-part (CIP) of U.S. application Ser. No. 12/052,296, filed on Mar. 20, 2008, which claims the benefit of U.S. Provisional Application No. 60/919,008, filed on Mar. 20, 2007, the contents for both of which are hereby incorporated by reference as if set forth fully herein.

### FIELD OF THE INVENTION

The present invention relates to devices and methods for providing consistent skin side temperature of a thermal device during use. The devices and methods of the invention provide a rate of change in temperature of less than about 0.8.

### BACKGROUND OF THE INVENTION

Disposable and reusable devices such as heat wraps have become a popular way to apply heat for relief of discomfort from temporary or chronic aches, pains, and injuries. Common heat wraps, for example, typically comprise a heat source containing an exothermic composition that generates heat, wherein the exothermic composition comprises metal powder, salts, and water and allows the exothermic composition to release heat upon oxidation of the metal powder. Other devices can be reusable and include solid, particulate or gel type materials that can be reheated and reused. Still other devices can be electric, either plugged into an electrical supply, or battery-operated. Devices incorporating such heat sources are generally found to be suitable for treatment of aches and pains associated with stiff muscles and joints, nerve pain, back pain, rheumatism, arthritis, and injuries, etc.

Such devices can provide sustained heat for periods from about one hour to about twenty-four hours, and are generally more convenient, portable, accessible, and affordable than, for example, whirlpools, hot towels, hydrocollators, and electric heating pads.

Most currently available heat devices are designed to generate a constant amount of heat versus produce a constant temperature. Such devices will produce or provide a consistent temperature only when the heat generation rate and the heat loss rate of the device remain constant. In use, the actual temperature of the heat source of the device, and consequently the temperature on the skin side of the device and thus on the skin of a user, can vary. Most such heat devices are manufactured to produce a certain temperature of the heat source under fixed conditions. They are typically tested by placing the device onto a constant temperature plate at a fixed ambient temperature. However, actual use conditions are often different. Particularly, the rate of heat removal can vary greatly. Heat removal during use is affected by various factors including the user's body's ability to dissipate heat (i.e. poor blood perfusion causing a reduction in the amount of heat the body can remove), and environmental conditions including clothing, ambient temperature, and airflow over the device. In addition, heat generation rate can be increased during wear due, for example, to body movement that increases air flow to an exothermic composition. As a result, the heat source temperature, and the temperature on the skin side of the device and the user's skin can reach temperatures exceeding 43° C., the temperature at which the skin can be burned. In addition, generally, such a rise in skin side temperature is gradual and not easily perceptible by the user even when it approaches or exceeds 43° C. Thus, injury can occur slowly, without the user noticing until it is too late and the skin has been burned.

Thus, there have been efforts to reduce or eliminate such burns. The majority of such approaches focuses either on regulating heat source temperature or regulating the amount of heat generation. To regulate the heat source temperature in exothermic compositions, many such efforts incorporate a phase change material in with the exothermic composition to absorb excess heat. However, phase change materials have a finite heat absorption capacity and are expensive. Thus, they would not be practical for heat wraps used for therapeutic pain relief where 8 hours or more of heat is required. Therefore preventing overly hot exothermic compositions can be difficult and/or expensive.

Other approaches to maintain a constant temperature using exothermic compositions include controlling the rate of reaction to compensate for any change in heat loss rate. This is typically done by adjusting the air flow through the device, or by releasing excess water into the heat generation chemistry for reducing oxygen accessibility in the reaction medium. However, in practice, controlling heat generation via controlling air flow is difficult, because for example body movement can affect air flow. Another approach has been to control the reaction by adding agents that will quench the reaction when the temperature exceeds a given maximum. However, high temperature during transportation and in storage conditions could prematurely release the quenching agents prior to use of the device and render the device ineffective.

Thus, there remains a need for an effective device and method to regulate skin side temperature of thermal devices in order to provide a device that provides sufficient heat generation in order to satisfy a user's desire for quickly perceived warmth. However, there also remains a need for a cost-effective means for delivering therapeutic heat while reducing or eliminating heat injury to the skin. Finally, there remains a need to provide for better fit, more consistent pressure distribution, and greater user comfort of single and/or multiple use thermal devices having single or multiple heat sources.

### SUMMARY OF THE INVENTION

The present invention includes devices that provide consistent skin side temperature comprising a primary insulative material disposed on a skin side of a thermal source; wherein the device provides a rate of change temperature of less than about 0.8.

In one embodiment, the present invention provides a method of delivering safe therapeutic heat to an unconscious or inert subject comprising:
applying a disposable device that provides a consistent skin side temperature to the unconscious or inert subject, wherein said device comprises:
   a thermal source comprising a particulate exothermic composition; and
   a primary insulative material disposed on a skin side of the thermal source that delivers heat while protecting the subject from burns;
   wherein said device provides a rate of temperature change of less than about 0.8.

In another embodiment, the device provides safe heat therapy to the subject while sleeping.

In another embodiment, said primary insulative material has a thermal conductivity of about 0.045 to 0.055 W/m−k.

The present invention also provides and maintains a skin side temperature of less than about 43° C., over a wide range of use conditions, in order to eliminate heat induced injury to the skin.

The present invention provides and maintains a skin side temperature of from about 36° C. to about 42° C. for up to about 24 hours.

The present invention also includes methods of providing consistent skin side temperature, methods of improving skin health and methods of providing passive skin side temperature control, by applying to a user's skin devices comprising a primary insulative material disposed between a thermal source and a user's skin; wherein the device provides a rate of change in skin side temperature to change in thermal source temperature of less than about 0.8. i.e. for every change of 1° C. of a thermal source temperature, there is a change of less than about 0.8° C. in the skin side temperature of the device.

### BRIEF DESCRIPTION OF THE DRAWINGS

FIG. 1 is a schematic diagram of an embodiment of the invention having a primary insulative material on a skin side of a thermal source.

FIG. 2 is a schematic diagram of an embodiment of the invention having a primary insulative material on a skin side of a thermal source, a top layer, and a skin side layer.

FIG. 3 is a schematic diagram of an embodiment of the invention having a primary insulative material on the skin side of a thermal source and a second insulative material on the top side of the thermal source, facing away from the skin.

FIG. 4 is a schematic diagram of an embodiment of the invention having a primary insulative material on a skin side of a thermal source and a top layer on the top side of a thermal source.

FIG. 5 is a schematic diagram of an embodiment of the invention wherein layers of material form the primary insulative material.

FIG. 6 is a plan view of an embodiment of the thermal device of the present invention.

FIG. 7 is a partial sectioned side elevational view of the embodiment shown in FIG. 6.

### DETAILED DESCRIPTION OF THE INVENTION

The present invention includes devices and methods that provide consistent skin side temperature comprising: a primary insulative material disposed on a skin side of a thermal source; wherein said device provides a rate of change temperature of less than about 0.8. i.e. for every change of 1° C. in a thermal source temperature, there is a change of less than about 0.8° C. in the skin side temperature of the device.

The term “inert” or “inert subject” as used herein refers to a subject that has a diminished capacity to respond to sensory stimuli, such as heat. Thus an inert subject would not be responsive to applied heat having the tendency to burn. An inert subject includes one that is asleep or unconscious as well as one who is conscious, but due to some other sort of incapacity, such as paralysis or perception deficit, is incapable of addressing or responding to potentially damaging external sources, such as heating capable of burning skin. Preferably, an inert or unconscious subject is one that is sleeping.

Safe application of the devices described herein, refers to application of the devices to skin of a subject without resultant burns or any other serious skin damage.

### Devices

Non-limiting examples of devices of the present invention include single use, disposable, and reusable coverings, wraps, and/or devices for the neck, shoulder, back, abdomen, hand, wrist, elbow, arm, leg, knee, and ankle, wherein such devices conform to the contours of a user's body. Devices of the present invention can be worn directly contacting the user's skin, or over clothing. Hereinafter use of the word “skin” in reference to the device contacting, being next to, or disposed on, a user's skin means directly contacting the skin and/or facing the skin if the device is worn over clothing.

The devices of the present invention provide rate of temperature change in skin side temperature to thermal source temperature of less than about 0.8. Thus, for every change of 1° C. of a thermal source temperature, whether an increase or a decrease, there is a change of less than about 0.8° C. in skin side temperature of the device. Alternatively, the devices provide a rate of change in skin side temperature of the device to change in thermal source temperature of less than about 0.7, and alternatively of less than about 0.5. The rate of temperature change is determined according to the method described herein.

The devices and methods of the present invention also consistently maintain a skin side temperature of below about 43° C. Alternatively, the devices and methods of the present invention maintain a consistent skin side temperature from about 36° C. to about 42° C., alternatively from about 37° C. to about 42° C., and alternatively from about 39° C. to about 42° C.

In addition, the devices and methods attain and provide a skin side temperature of at least about 36° C. in about 5 minutes.

The devices of the present invention comprise a primary insulative material disposed at a skin side of a thermal source. The devices can additionally comprise at least one top layer disposed over the thermal source at a top side of the thermal source, and at least one optional skin side layer disposed between the primary insulative material and a user's skin or clothing. Such a top and/or optional skin side layer(s) can be provided for various reasons and uses including, look, feel, comfort, color, shape, fit, and combinations thereof. The devices can also comprise a second insulative material disposed at a top side of the thermal source, facing away from the user's skin, disposed between the thermal source and the top layer.

In addition to providing better control of skin side temperature than previously possible, the devices and methods of the present invention also provide better fit and conformation of devices for delivering heat. In particular, if a plurality of heat cells, as described below, is used, the plurality of heat cells aid in achieving better fit of the thermal device, by allowing the device to be flexible and bendable in various directions or axes. Thus, the present invention provides better overall skin health by reducing or eliminating not only heat injury to the skin, but also pressure, friction, or slippage-induced injury due to devices that are too tight or too loose and do not remain in place well.

### Primary Insulative Material

As used herein, primary insulative material means insulative material employed in addition to any top layer, skin side layer, and/or thermal source used to form a device. The primary insulative material provides the primary temperature change effect described herein.

The control of skin side temperature is important to avoid heat related injury to the skin. However, for a user to feel the effects of a thermal device there must be a noticeable temperature increase after the device is put on the user's skin. Normal human body temperature is approximately 35° C. Thus, temperatures above about 35° C. are typically perceived as warm to the skin. However, temperatures above about 43° C. can burn the skin. Because use conditions of thermal devices vary, and thus affect the skin side temperature of a thermal device, in certain use conditions thermal source temperature and skin side temperature of some devices can rise well above 43° C.

With the present invention, a user of the device can quickly sense warmth because the present invention attains and provides a skin side temperature of about 36° C. within about 5 minutes after initiation of heating. However, using primary insulative material in a thermal device at the skin side of a thermal source allows for target skin side temperature of about 39° C. to about 42° C. to be reached without burning the user's skin. A thermal source temperature usable in the present invention need only be about 2° C. to about 3° C. higher than the desired skin side temperature. In addition, regardless of thermal source temperature above 43° C. and use conditions, the skin side temperature of the device of the present invention does not exceed about 43° C. during the use period. The skin side temperature of the devices of the present invention warms to the target skin side temperature of about 39° C. to about 42° C. within about 30 minutes of initiation of heating, and alternatively within about 15 minutes of initiation of heating.

The skin side temperature can be maintained in the target temperature range for a time period of greater than about 1 hour, alternatively greater than about 4 hours, alternatively greater than about 8 hours, alternatively greater than about 12 hours, alternatively greater than about 16 hours, and alternatively for about 24 hours. The devices and methods of the present invention can provide sustained pain relief for at least about 2 hours, alternatively for about 8 hours, alternatively for about 16 hours, alternatively for about 1 day, and alternatively for about 3 days after the device is removed from the user.

Thus, the addition of a primary insulative material on the skin side of a thermal source dampens the effects of changes (up or down) in thermal source temperature in order to provide a consistent skin side temperature of the device in the desired temperature range throughout an extended time period.

The primary insulative material of the present invention incorporates air to provide its insulative properties. Air is nature's most effective insulator. In order to maintain its insulative properties during use, another desirable property of the insulative material is that it does not collapse when subjected to pressure normally encountered during use, such as laying on one's back while wearing a back wrap.

Non-limiting examples of suitable materials for the primary insulative materials include: foam such as open-cell foam and/or closed cell foam, non-woven, sponge, glass wool, fiberglass. The materials can be spunbond, meltblown, carded, hydroformed, foamed thermoformed, spray-on, air laid and combinations thereof as would be understood by those of skill in the art. Insulative materials that are particularly useful include, for example, polypropylene microfoam MF060 available from Pregis Corp., Deerfield, Ill., USA, and polyethylene astrofoam also available from Pregis— 1/32″ (AF030), 1/16″ (AF060), 1/32″ (AF090).

In a preferred embodiment, the primary insulative material is polyethylene or polypropylene foam having a thickness of about 1/16″, a density of about 0.5-1.5 pcf, more particularly 1.15 pcf, a tensile strength (psi) of about 1-100 min, and elongation percent of less than 95, more preferably about 50-60%. In another preferred embodiment, the primary insulative material has a thermal conductivity of about, 0.02 to 0.08 W/m−k, more preferably, 0.03 to 0.07 W/m−k, most preferably 0.04 to 0.06 W/m−k.

Alternatively, for example, layers of one or more types of non-woven materials can be used as the primary insulative material. A plurality of layers of non-woven material can be folded, reformed or stacked, and bonded together to increase thickness such that a non-woven material can itself be the primary insulative material disposed between the thermal source and the user's skin or clothing. The layers can be bonded, as a non-limiting example as would be understood by one of skill in the art, with a construction adhesive such as #70-4589 available from National Starch & Chemical Company, Bridgewater, N.J., USA. Alternatively, non-woven materials can be bonded to each other ultrasonically, thermally, or by other means known to those of skill in the art of bonding non-woven materials. Non-limiting examples of suitable non-woven materials include: nylon, rayon, cellulose ester, polyvinyl derivatives, polyolefins, polyamides or polyesters, polypropylene, cuproammonium cellulose (available from Asahi Kasei America Inc., New York, N.Y., USA) and other high molecular weight compounds, as well as natural materials including wool, silk, jute, hemp, cotton, linen, sisal, and ramie. Non-limiting examples of particular suitable non-woven materials include; polypropylene carded non-woven #6780 available from PGI, Inc., Waynesboro, Va., USA, at 22 grams per square meter (gsm); or highly elongated carded non-woven available from Fiberweb, Simpsonville, S.C. USA; or a 30 gsm SMMS (spunbond, meltblown, meltblown, spunbond) laminate such as from First Quality Nonwovens, Inc. (FQN), Great Neck, N.Y., USA. The non-woven materials can be spunbond, meltblown, carded, highly elongated carded, air laid, and combinations thereof, as would be understood by those of skill in the art. Such non-woven materials are generally described in Riedel “Nonwoven Bonding Methods and Materials”, Nonwoven World, (1987).

The primary insulative material has a thickness of from about 1/64 inch to about 1 inch to provide the requisite insulative properties, depending on the type of primary insulative material, the type of thermal source used, and the desired skin side temperature. Alternatively, the primary insulative material has a thickness of from about 1/32 inch to about ½ inch, and alternatively from about 1/16 inch to about ⅜ inch. A particularly useful thickness and type of primary insulative material is 1/16 inch thick polypropylene microfoam MP060 available from Pregis Corp., Deerfield, Ill., USA.

If foam is used as the primary insulative material and/or as a second insulative material, e.g. at the skin side and/or top side of a thermal source, such foam can conform to the contours of a user's body and thus provide improved delivery of heat. Thus, the combination of a thermal source comprising a plurality of heat cells, and the primary insulative material enables the thermal device to easily conform to body contours which reduces the need to overtighten the device in an attempt to achieve a secure lit.

The primary insulative layer can be bonded to, as a non-limiting example, a thermal source containing an exothermic composition. Such bonding can be, as a non-limiting example as would be understood by one of skill in the art, with a construction adhesive such as #70-4589 available from National Starch & Chemical Company, Bridgewater, N.J., USA. Alternatively, such bonding can be ultrasonically, thermally, or by other means known to those of skill in the art.

The primary insulative material can be covered by an optional skin side layer of material between the primary insulative material and the user's skin or clothing.

### Thermal Source

A thermal source usable with the present invention can be a single use thermal source, a reusable or multi-use thermal source, an electrical thermal source, an exothermic composition thermal source, heat of crystallization composition thermal source, and combinations thereof.

An example thermal source useful in the present invention comprises at least one heat cell comprising an exothermic composition. The at least one heat cell is formed in a unified structure comprising at least two opposing surfaces, wherein at least one surface is air permeable, and wherein the exothermic composition is filled between the two opposing surfaces.

In an embodiment of a heat cell, the two opposing surfaces can be film layer substrate surfaces. The film layer substrate surfaces can be made of films or films laminated to non-woven materials. Generally preferred films are those that are heat sealable and capable of being easily thermally fused. Non-woven materials, if used, can provide support and integrity to the film layer substrates.

Non-limiting examples of suitable films include polyethylene, polypropylene, nylon, polyester, polyvinyl chloride, polyvinylidene chloride, polyurethane, polystyrene saponified ethylene-vinyl acetate copolymer, ethylene-vinyl acetate copolymer, natural rubber, reclaimed rubber, and synthetic rubber. Preferable film layer thickness is in the range of about 1 to about 300 μm and can be air permeable or impermeable.

Preferred non-woven materials have characteristic properties of being light weight and having high tensile strength. Non-limiting examples include: nylon, rayon, cellulose ester, polyvinyl derivatives, polyolefins, polyamides or polyesters, polypropylene, cuproammonium cellulose (available from Asahi Kasci America Inc., New York, N.Y., USA) and other high molecular weight compounds, as well as natural materials including wool, silk, jute, hemp, cotton, linen, sisal, and ramie. Non-limiting examples of particular suitable non-woven materials include; polypropylene carded non-woven #6780 available from PGI, Inc., Waynesboro, Va., USA, at 22 grams per square meter (gsm); or highly elongated carded non-woven available from Fiberweb, Simpsonville, S.C., USA; or a 30 gsm SMMS (spunbond, meltblown, meltblown, spunbond) laminate such as from First Quality Nonwovens, Inc. (FQN), Great Neck, N.Y., USA. The non-woven materials can be spunbond, meltblown, carded, highly elongated carded, air laid, and combinations thereof as would be understood by those of skill in the art. Such non-woven materials are generally described in Riedel “Nonwoven Bonding Methods and Materials”, Nonwoven World, (1987).

Preferred film layer substrate surfaces include polypropylene (PP) non-woven sheets laminated to a film of poly(ethylene vinyl acetate) (EVA) or low density polyethylene (LDPE) having a total thickness, of the combination of all of the layered materials such as film, plus any construction adhesive, plus non-woven, of about 400 to about 1500 μm. An example of a commercially available non-woven sheet useful with the present invention is material No. W502FWH, available from PGI (Polymer Group International) located in Waynesboro, Va., USA. An example of a commercially available polypropylene/ethylene vinyl acetate (PP/EVA) film material useful with the present invention is No. DI-1245, available from Clopay Plastics of Cincinnati, Ohio, USA.

The two opposed surfaces can be created by bonding two film layer substrate surfaces together around their peripheries thereby forming a pouch, recess, envelope, pocket, or chamber. The film side of each material is toward the inside of the pouch, recess, envelope, pocket, or chamber (i.e. the side to be filled) and the non-woven side is toward the outside. Pouches can also be made in the film layer substrate surfaces by thermoforming, mechanical embossing, vacuum embossing, or other means. A preferred method is by thermoforming, such as that described in “Thermoforming”, The Wiley Encyclopedia of Packaging Technology, pp. 668-675 (1986), Marilyn Bakker, ed.

The resulting heat cell can have any geometric shape, including but not limited to disk, triangle, pyramid, cone, sphere, square, cube, rectangle, rectangular parallelepiped, cylinder, and ellipsoid.

The air permeability of the heat cells can be provided by selecting films or film coatings for the film layer substrate surfaces forming and/or covering the pouches, recesses, envelopes, pockets or chambers. The desired permeability can be provided by microporous films or by impermeable films which have pores or holes formed therein. The formation of such pores or holes can be via extrusion cast/vacuum formation or by hot needle aperturing. Air permeability can also be provided by perforating at least one of the film layer substrate surfaces with aeration holes using, for example, at least one pin, preferably an array of from about 20 to about 60 pins. Although there are preferably provided aeration holes in the upper film layer substrate surface, it is also possible to provide aeration holes in the lower film layer substrate surface, and/or provide aeration holes in both.

Non-limiting examples of permeability usable with the present invention include air permeability (measured using methods described in ASTM D737-96) of less than about 4 cfm, alternatively less than about 3 cfm, alternatively less than about 2 cfm, alternatively less than about 1.5 cfm, and alternatively less than about 0.8 cfm.

The heat cells containing the exothermic composition can be generally prepared by constructing pockets in film layer substrate surfaces such as a polypropylene/ethylene vinyl acetate layer; constructing a particulate exothermic premix; adding a fixed amount of particulate exothermic premix into each pocket; rapidly dosing the particulate exothermic premix with a brine solution to form an exothermic composition; placing a flat sheet of a polypropylene/ethylene vinyl acetate film layer substrate surface over the filled pockets with the ethylene vinyl acetate side facing the ethylene vinyl acetate side of the preformed pocket-containing layer. The two film layer substrate surfaces are bonded together using a low heat, thereby forming a unified structure containing a plurality of heat cells. A plurality of apertures can be formed in the polypropylene/ethylene vinyl acetate film layer substrate surface forming and/or covering the filled pockets such that one or both of the film layer substrate surfaces is made air permeable. Non-limiting examples of components of a particulate exothermic premix composition usable with the present invention include iron powder, carbon, absorbent gelling material, and water. Non-limiting examples of components of a brine solution include a metal salt, water, and optionally a hydrogen gas inhibitor such as sodium thiosulfate.

The velocity, duration, and temperature of the thermogenic oxidation reaction of the exothermic composition can be controlled as desired by regulating the amount of air available for the oxidation reaction of the exothermic composition. More specifically, one can change the air diffusion and/or permeability through the air permeable film layer substrate surface(s) such as by varying the degree of perforation of the film layer substrate surface(s) and providing a plurality of apertures through the film layer substrate surface(s). Other methods of modifying the exothermic reaction include but are not limited to the choice of components of the heat source, for example by choosing a specific component or modifying component particle size. Additionally, the porosity of the exothermic composition for air diffusion can be varied, for example, by the inclusion of a water manager such as vermiculite and/or water absorbent gelling agents such as sodium polyacrylate, and by varying the amount of brine added.

The resultant unitary structure comprising the plurality of heat cells can be used alone or can be incorporated into variously sized and shaped thermal devices such as disposable and reusable wraps. Typical wrap devices can have a means for retaining the device on the desired body location. Non-limiting examples of such means include, straps with hook and loop type closures and/or adhesives.

Thermal sources comprising air activated exothermic composition heat cells as described above are preferably packaged in secondary air-impermeable packaging to prevent the oxidation reaction from occurring until desired.

Alternatively, air impermeable removable strips such as adhesive strips can be placed over the perforations in the air permeable film layer substrate surface such that when the strips are removed, air enters the heat cells, thus activating the oxidation reaction. Alternatively, instead of being integrally incorporated into a thermal device, heat cells can be formed into a separate thermal source structure such as a sheet or layered structure that can be disposable and releasably attachable to a wrap device that can be reusable.

### Top Layer

The optional top layer of embodiments of the present invention can be a non-woven material. Non-limiting examples of suitable materials for the top layer include: nylon, rayon, cellulose ester, polypropylene, polyvinyl derivatives, polyolefins, polyamides, or polyesters, cuproammonium cellulosic fiber (available from Asahi Kasei America Inc., New York, N.Y., USA) and other high molecular weight compounds, as well as natural materials including wool, silk, jute, hemp, cotton, linen, sisal, and ramie. Non-limiting examples of particular suitable non-woven materials include; polypropylene carded non-woven #6780 available from PGI, Inc., Waynesboro, Va., USA, at 22 grams per square meter (gsm); or highly elongated carded non-woven available from Fiberweb, Simpsonville, S.C., USA; or a 30 gsm SMMS (spunbond, meltblown, meltblown, spunbond) laminate such as from First Quality Nonwovens, Inc. (FQN), Great Neck, N.Y., USA. The non-woven materials can be carded, highly elongated carded, spunbond, meltblown, air laid and combinations thereof as would be understood by those of skill in the art. Such non-woven materials are generally described in Riedel “Nonwoven Bonding Methods and Materials”, Nonwoven World, (1987). The top layer can be disposed over the thermal source on a top side of the thermal source, and can be applied, as a non-limiting examples as one skilled in the art would understand, with a hot melt construction adhesive such as # 70-4589 available from National Starch & Chemical Company, Bridgewater, N.J., USA and spirally applied at 15 grams per square meter (gsm). Alternatively, such bonding can be ultrasonically, thermally, or by other means known to those of skill in the art.

### Optional Skin Side Layer

The device of the present invention can also comprise an optional skin side layer disposed over the primary insulative material, such that it is between the primary insulative material and the user's skin or clothing. The optional skin side layer can be attached using, as a non-limiting example one skilled in the art would understand, a hot melt construction adhesive such as # 70-4589 available from National Starch & Chemical Company, Bridgewater, N.J., USA and spirally applied at 15 grams per square meter (gsm). Alternatively, such bonding can be ultrasonically, thermally, or by other means known to those of skill in the art.

The optional skin side layer can be, for example, a layer of non-woven material. Non-limiting examples of materials suitable for an optional skin side layer include: nylon, rayon, cellulose ester, polyvinyl derivatives, polyolefins, polypropylene, polyamides or polyesters, cuproammonium cellulose (available from Asahi Kasei America Inc., New York, N.Y., USA) and other high molecular weight compounds, as well as natural materials including wool, silk, jute, hemp, cotton, linen, sisal, and ramie. Non-limiting examples of particular suitable non-woven materials include: polypropylene carded non-woven #6780 available from PGI, Inc., Waynesboro, Va., USA, at 22 grams per square meter (gsm), a highly elongated carded non-woven available from Fiberweb, Simpsonville, S.C., USA, or an SMMS (spunbond, meltblown, meltblown, spunbond) laminate such as a 30 gsm SMMS laminate from First Quality Nonwovens, Inc. (FQN), Great Neck, N.Y., USA. The non-woven materials can be carded, highly elongated carded, spunbond, meltblown, air laid and combinations thereof as would be understood by those of skill in the art. Such non-woven materials are generally described in Riedel “Nonwoven Bonding Methods and Materials”, Nonwoven World, (1987).

### Second Insulative Material

In addition to a primary insulative material, a second insulative material can be disposed between the thermal source and the top layer. The second insulative material can be the same or different material as the primary insulative material, and can be chosen from the materials described above for the primary insulative material. However, if a second insulative material is used on the top side of the device and the top side of the device is air-permeable, the second insulative material must be, or be made, air permeable in order for air to enter the device to activate the exothermic composition if an air activated exothermic composition is used as the thermal source of the device. The second insulative material can be attached, as a non-limiting example, to the top film substrate surface of an exothermic thermal source and/or to the top layer by, as a non-limiting example as one of skill in the art would understand, a hot melt construction adhesive such as # 70-4589 available from National Starch & Chemical Company, Bridgewater, N.J., USA and spirally applied at 15 grams per square meter (gsm). Alternatively, such bonding can be ultrasonically, thermally, or by other means known to those of skill in the art.

### Attachment Means

The devices of the present invention can have various means for retaining the devices on or around various body parts such as neck, shoulder, back, abdomen, hand, wrist, arm, elbow, leg, knee, and ankle. Non-limiting examples of such means include at least one strap which can be flexible and/or elastomeric, adhesive, hook and loop fastening systems, and combinations thereof.

### Optional Components

The devices for regulating skin temperature of the present invention are particularly advantageous for optionally incorporating a component to be released from the device. Non-limiting examples of such components include aromatic compounds, skin treatment compounds, pharmaceutical or therapeutic agents, and mixtures thereof. The optional component can be incorporated into heat cells as a separate layer; incorporated into at least one of the film layer substrates (described above) of at least one heat cell; incorporated into the primary insulative material; and/or incorporated into the top layer and/or the skin side layer. Non-limiting examples of such components include menthol, camphor, eucalyptus, and combinations thereof; benzaldehyde, citral, decanal, aldehyde, and combinations thereof; antibiotics, vitamins, skin treatment and/or softening and/or conditioning compositions, antiviral agents, antifungal agents, analgesics, anti-inflammatory agents, antipruritics, antipyretics, anesthetic agents, antimicrobial agents, and combinations thereof. The devices can also comprise a self-adhesive component and/or a sweat-absorbing component incorporated as a separate layer, or incorporated into at least one of the film layer substrates of one or more heat cells.

### Rate of Temperature Change Method

As used herein the “rate of temperature change” provided by a device is the rate of change in skin side temperature of a device to thermal source temperature, measured as described below at fixed test conditions. Devices and methods of the present invention provide a rate of temperature change of less than about 0.8. i.e. for every change of 1° C. in a thermal source temperature, there is a change of less than about 0.8° C. in the skin side temperature of the device.

Devices to be measured are measured in a temperature controlled room that is maintained at 23° C.−/−0.2° C. There should be no air flow over the temperature measurement site in the room.

When measuring a device, the rate of temperature change results from the device as a whole, excluding the thermal source. Most materials that are found in thermal devices, such as those of a top layer and a skin side layer as described above, have some insulative properties. When measuring the rate of temperature change of a thermal device, the device must cut apart and the thermal source removed—i.e. any exothermic composition, particulate composition, gel type composition or electrical elements must be removed from the device being tested.

The rate of temperature change of a device is measured by taping the device, (with its thermal source removed) on to the surface of a hot plate with the skin side of the device facing away from the surface of the hot plate. The hot plate used is a laboratory hot plate with a variable temperature control setting, model SP 49625 (or equivalent model) made by Barstead/Thermolyne, Dubuque, Iowa, USA. To increase the amount of control of the heating of the hot plate, the hot plate is connected to a Powerstat variable autotransformer manufactured by Werner Electric, Bristol, Conn., USA.

The device to be tested is taped to the hot plate with 3M 6200 tape, manufactured by 3M Company, St. Paul, Minn., USA. It is important that the device maintains good even contact with the surface of the hot plate. Thus, the edges of the device are taped to the surface of the hot plate. Care is taken not to stretch or deform the device in any way. The 3M 6200 tape is used because it is flexible and stretchable and provides the required tension to assure the device is maintained flat and in good even contact with the surface of the hot plate without stretching or deforming the device.

To measure the temperature of the surface of the hot plate, a thermocouple, type K insulated beaded wire thermocouple, part number SC-GG-K-30-36, having a diameter of 0.010 inches, from Omega Engineering, Inc., Stamford, Conn., USA is used. The thermocouple is taped to the surface of the hot plate, using the 3M 6200 tape, at a location on the surface of the hot plate adjacent to the location where the thermal device is taped.

To measure the temperature of the skin side of the device, a type K thermocouple is taped to the skin side of the device, using 3M 6200 tape.

Both thermocouples are attached to one handheld thermometer, model HH84, manufactured by Omega Engineering, Inc., Stamford, Conn., USA.

To test the rate of temperature change of a device, the hot plate is set at a stable temperature of 50° C. as measured by the thermocouple that is taped to the surface of the hot plate. The hot plate is calibrated by the manufacturer. The hot plate is allowed to stabilize for 15 minutes. Once the temperature of the hot plate has stabilized, the device to be tested is taped to the hot plate. The second thermocouple is then taped to the device as described above, and connected to the HH84 meter. Both thermocouples are attached to the HH184 thermometer. The HH84 is a dual channel meter so only one meter is needed. The HH84 thermometer is pre-calibrated by the manufacturer and set to acquire and record data when the ‘on’ button is pushed. The power to the hot plate is then turned off and the data acquisition feature of the HH84 thermometer is turned on. The HH84 thermometer has a data acquisition capability and is programmed to record temperature measurements every 3 seconds for at least 1 hour for the two thermocouples attached to it. The data from the HH84 thermometer is then transferred to a computer where it is tabulated in an Excel spreadsheet (Excel software available from Microsoft Corporation, Redmond, Wash., USA). As the hot plate cools, the temperature difference between the surface of the hot plate and surface of the skin side of the thermal device is recorded over time. Due to the thermal mass of the hot plate the cooling rate is slow enough to enable steady temperature readings to be made on the hot plate surface and the thermal device.

### Calculations

The devices and methods of the present invention provide that a rise or fall in thermal source temperature results in a smaller (less than one for one) rise or fall in skin side temperature of a thermal device. Once the temperature measurements have been recorded, a least squares linear regression analysis of the skin side temperature of the thermal device against its corresponding hot plate temperature is performed, using Sigma Plot software from Systat Software Inc, San Jose, Calif., USA, to fit the equation T_{TD}=m×T_{HP}+b. The variables T_{TD} and T_{HP} represent the temperatures of the skin side of the thermal device (TD), and temperature of the surface of the hot plate (HP) respectively. m is the slope of the line, and b is the T_{TD} axis intercept at the point where T_{HP} is equal to zero. The rate of temperature change of the tested device is reflected in the slope of the line, m. The slope, m, of the line represents how the skin side temperature of the tested device changes with respect to changes in thermal source temperature. The devices and methods of the present invention provide a slope, m, of less than about 0.8. A least squares analysis is a standard statistical analysis performed on data point to fit a straight line to the data points. Such analysis is described in “Statistics for Experimenters”, George E. P. Box, William G. Hunter, J. Stuart Hunter, published by John Wiley & Sons, Inc. (1978), 453-455.

### Methods of Making

Devices according to the present invention can comprise a number of different embodiments.

An embodiment of a device of the present invention can be made by forming a unitary sheet structure having a plurality of heat cells, made as described above, as a thermal source; cutting the sheet structure into the desired device shape then attaching, with a construction adhesive, one or more layers, such as a primary insulative material, a top layer, and/or an optional skin side layer. The primary insulative material can be positioned between the thermal source and an optional skin side layer, or can directly contact the user's skin or clothing, as described above.

If an exothermic composition is used as the thermal source, preferably at least one of the film layer substrate surfaces enclosing the exothermic composition is air permeable and is preferably disposed at the top of the thermal source, facing away from the skin side of the device. However, if only one of the film layer substrate surfaces is air permeable, and that air permeable side of the thermal source is placed toward the skin of the user, the air permeability can be adjusted to still allow air to reach the exothermic composition. For example, the aeration holes can be adjusted in size to be large enough such that the air flow into the thermal source is not rate limiting. Alternatively, the porosity of the exothermic composition can be modified to be the rate limiting step. However, preferably the air permeable film layer substrate surface is at the top side of the thermal source, facing away from the skin side of the device.

### Uses

It is preferred that the device described herein is applied to inert or unconscious subjects. In particular, methods of delivering safe therapeutic heat to an unconscious or inert subject are provided, comprising: applying a disposable device as described herein, which provides a consistent skin side temperature to the unconscious or inert subject are provided.

In one embodiment, the device is used in an “overnight” context, wherein it is applied to a subject before going to sleep and provides consistent and safe heat throughout the night. Because the subject is asleep during application of the device it is particularly important that the device provide consistent heating and avoid temperature spikes or elevations in temperature that could cause burns to the subject.

Additionally, because the unconscious or inert subjects may not be aware of what position they are in relation to the heating device, it is important that the device not be susceptible to compression effects, which in prior art devices can either increase the heat due to the proximity of the exothermic composition to the user or result in decreased efficacy and heating from quenching of oxygen flow.

Through utilization of the particular primary insulative layer and associated configuration in the claimed disposable device, applicants have created a device that is resistant to such compression effects and resultant inconsistent heating profiles. Notably, because the layers between the user and the exothermic composition are resistant to compression from body pressure, they do not result in significant quenching or excess heating.

Accordingly, the device is particularly suited for users who are unconscious or inert, such as a sleeping subject, who is prone to rolling over or moving in sleep and unaware of the resultant effects such movement would have on the heating device.

### EXAMPLES

The following non-limiting examples further describe and demonstrate embodiments within the scope of the present invention. The examples are given solely for the purpose of illustration and are not to be construed as limitations of the present invention, as many variations thereof are possible without departing from the spirit and scope of the present invention.

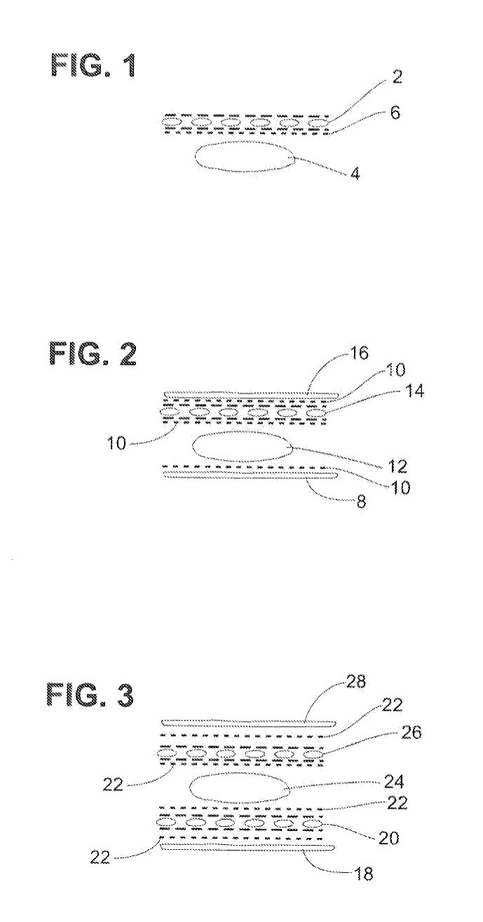

The schematic illustrations of FIGS. 1-5 are used only to represent the arrangement and order of components that can form the devices of the present invention. They are not drawn to scale. All thermal sources of FIGS. 1-5 are shown as an oval shaped area which is used for illustrative purposes only and does not represent any particular thermal source, nor proportional size or shape of any such thermal source.

FIG. 1 is a schematic illustration of an embodiment of the present invention in which a primary insulative material 2 is disposed on a skin side of a thermal source 4, and attached thereto by construction adhesive 6.

FIG. 2 is a schematic illustration of an embodiment of the present invention in which a primary insulative material is positioned between a skin side layer and a thermal source. A layer of foam, non-woven, spunbond, carded, hydroformed or airlaid insulative material is used as a primary insulative material between a thermal source and the skin side layer of a thermal device. The embodiment has a top side layer 8, attached by a construction adhesive 10 (i.e. hot melt construction adhesive # 70-4589 available from National Starch & Chemical Company, Bridgewater, N.J., USA) spirally applied at 15 grams per square meter (gsm) to a thermal source 12. A primary insulative material 14 is attached by construction adhesive 10, to the thermal source 12 and skin side layer 16.

FIG. 3 is a schematic illustration of an embodiment of the present invention wherein a primary insulative material is incorporated between a skin side layer and a thermal source, and a second insulative material is incorporated between a top side layer and the thermal source. The insulative materials used on the skin side and the top side of the thermal source can be the same or different materials. The device has a top layer 18, and a second insulative material 20, such as foam, non-woven, spunbond, carded, hydroformed, or airlaid insulative material. The second insulative material 20 is attached to the top layer 18 by construction adhesive 22 (i.e. # 70-4589 available from National Starch & Chemical Company, Bridgewater, N.J., USA). Second insulative material 20 is attached by construction adhesive 22, to a thermal source 24. Attached, by construction adhesive 22, to a skin side of the thermal source 24, is primary insulative material 26. Primary insulative material 26 is attached by construction adhesive 22, (i.e. # 70-4589 available from National Starch & Chemical Company, Bridgewater, N.J., USA), to a skin side layer 28.

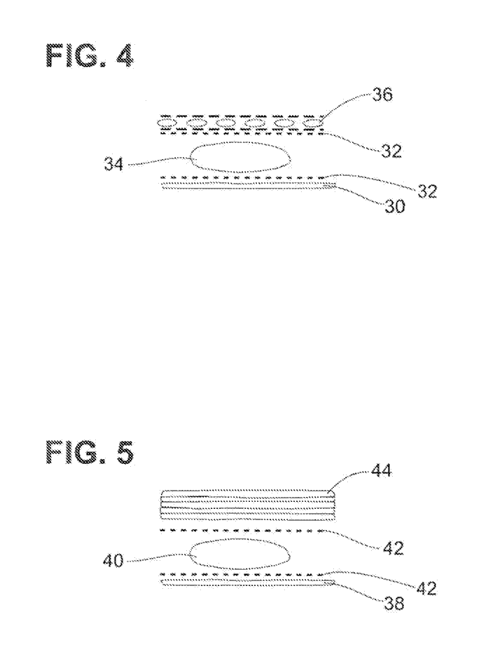

FIG. 4 is a schematic illustration of an embodiment in which a primary insulative material is attached to a skin side of a thermal source, and also forms the skin side layer of the thermal device. A top layer 30 is attached, by construction adhesive 32 (i.e. # 70-4589 available from National Starch & Chemical Company, Bridgewater, N.J., USA), to a thermal source 34. A primary insulative material 36 is attached to a skin side of the thermal source 34, with construction adhesive 32 and forms the skin side layer of the thermal device.

FIG. 5 is a schematic illustration wherein the primary insulative material is a plurality of layers of non-woven material which are folded, reformed or stacked and bonded together to increase thickness such that a non-woven material forms the primary insulative material, and which also forms the skin side layer of the thermal device. The layers are bonded with construction adhesive (i.e. #70-4589 available from National Starch & Chemical Company, Bridgewater, N.J., USA). The device has a top layer 38, a thermal source 40 attached to a top side thereof with construction adhesive 42. Construction adhesive 42 attaches the primary insulative material 44 to the skin side of the thermal source 40.

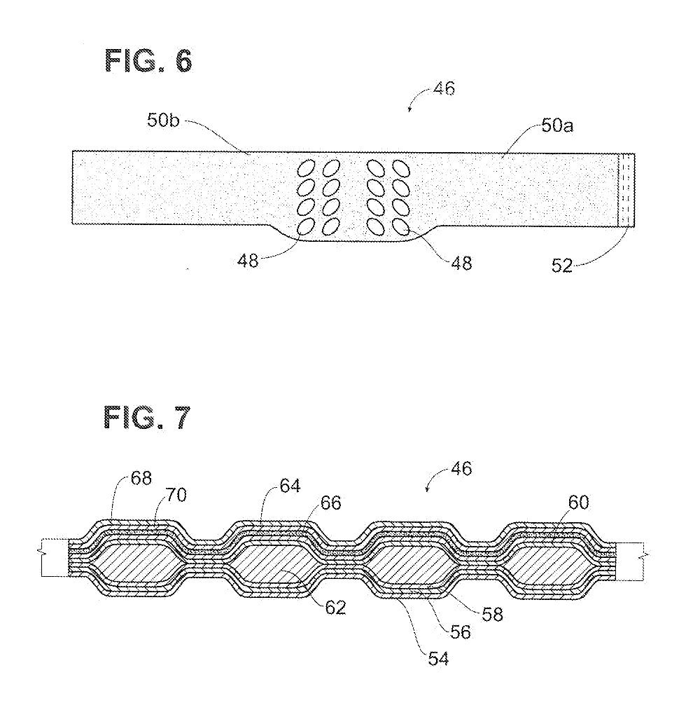

FIG. 6 a plan view of an embodiment of the thermal device of the invention for use on a user's back and/or hip area. The device 46 has a plurality of heat cells 48, and has extending strap portions 50a, 50b which are wrappable around a user's torso and/or hips. Attachment means 52 (i.e. one portion of a hook and loop fastening system) is shown at one end of strap portion 50a. Attachment means 52 is used to secure strap portions 50a, 50b together to retain the device around a user's torso and/or hips. A corresponding attachment means is disposed on strap 50b but is not visible in the view shown.

FIG. 7 is a partial sectioned view of the embodiment shown in FIG. 6. The device 46 comprises a top layer 54 attached, by construction adhesive 56, to a thermal source comprised of first and second film layers 58 and 60 which enclose exothermic composition 62. A primary insulative layer 64 is attached to film layer 60 by construction adhesive 66. A skin side layer 68 is attached to primary insulative layer 64 by construction adhesive 70.

### Conductivity Testing:

The thermal conductivity of various materials for use in the primary insulative layer described herein were tested at various temperatures to find an ideal candidate that had both good insulative properties to protect against burns, but still allowed adequate consistent heat to be delivered to the user. The results are summarized in Table 1.

TABLE 1

| | thickness | | | |
|---|---|---|---|---|
| | @ 25° C. | temperature | | conductivity λ |
| Sample | (mm) | (° C.) | (° F.) | (W/m-K) |
| Pregis Polypropylene | 1.24 | 33 | 91 | 0.046 |
| Foam | | 50 | 122 | 0.051 |
| | | 70 | 158 | 0.058 |
| Volara Polyethylene 2M | 2.63 | 40 | 104 | 0.034 |
| Foam | | 70 | 158 | 0.038 |
| Sealed Air Polyethylene | 1.46 | 40 | 104 | 0.048 |
| Foam | | 70 | 158 | 0.058 |
| LM Polyethylene Foam | 1.52 | 40 | 104 | 0.038 |
| | | 70 | 158 | 0.047 |
| Ivex Pro Foam | 1.51 | 40 | 104 | 0.060 |
| Polyethylene | | 70 | 158 | 0.075 |

The ideal conductivity was determined to be from about 0.045 to 0.055 W/m−k at about 40° C. This material has the beneficial effect of providing safe consistent heat through insulation to the user without substantially preventing heat from exiting the device.

In other embodiments, understandable by those of skill in the art, although not shown, devices that provide consistent skin side temperature can be formed into or as multi-use as well as single use devices depending on the thermal source used. Non-limiting examples of reusable and/or multi-use devices include those having at least one layer of at least one primary insulative material in a re-usable pocket that holds a thermal source, wherein the primary insulative material is disposed between the user's skin or clothing and the thermal source when used.

In additional embodiments, the primary insulative material(s) can be applied as a separable and/or removable part of a device or system that is applied directly to a user's skin, or used as part of a system, for example with a wrap device that holds a primary insulative material in place between a thermal source and a user's skin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of delivering safe therapeutic heat to an unconscious or inert subject comprising:
applying a disposable device that provides a consistent skin side temperature to the unconscious or inert subject, wherein said device comprises:
a thermal source comprising a particulate exothermic composition; and
a primary insulative material disposed on a skin side of the thermal source that delivers heat while protecting the subject from burns;
wherein said device provides a rate of temperature change of less than about 0.8.

2. The method of claim 1 wherein said primary insulative material has a thermal conductivity of about 0.045 to 0.055 W/m−k.

3. The method of claim 1, wherein said device provides a rate of temperature change of less than about 0.7.

4. The method of claim 2, wherein said device provides a rate of temperature change of less than about 0.5.

5. The method of claim 1, wherein said device provides and maintains a skin side temperature below about 43° C.

6. The method of claim 1, wherein said device provides and maintains a skin side temperature between about 39° C. and about 42° C.

7. The method of claim 1 wherein said device provides and maintains a skin side temperature between about 39° C. and about 42° C. for at least 5 hours.

8. The method of claim 1 wherein said primary insulative material is selected from the group consisting of: polyethylene or polypropylene foam.

9. The method of claim 1 wherein said primary insulative material has a thickness of from about 1/32 inch to about ⅜ inch.

10. The method of claim 1 wherein said thermal source comprises a plurality of heat cells comprising the particulate exothermic composition.

11. The method of claim 10 wherein said plurality of heat cells are formed in a unified structure comprising at least two opposed surfaces, wherein at least one surface is air permeable, and wherein said particulate exothermic composition is disposed between said two opposing surfaces.

12. The method of claim 1 further comprising a top layer disposed on a top side of said thermal source.

13. The method of claim 12 wherein said top layer is a non-woven material.

14. The method of claim 13 wherein said top layer is a material chosen from the group consisting of: nylon, rayon, cellulose ester, polyvinyl derivatives, polyolefins, polyamides, polyesters, polypropylene, celluloses, wool, silk, jute, hemp, cotton, linen, sisal, ramie, and combinations thereof.

15. The method of claim 1 further comprising a skin side layer.

16. The method of claim 15, wherein said skin side layer is a non-woven material selected from the group consisting of: nylon, rayon, cellulose ester, polyvinyl derivatives, polyolefins, polyamides, polyesters, polypropylene, celluloses, wool, silk, jute, hemp, cotton, linen, sisal, ramie, and combinations thereof.

17. The method of claim 15 wherein said primary insulative material is disposed between said thermal source and said skin side layer.

18. The method of claim 1 further comprising a second insulative material disposed on a top side of said thermal source.

19. The method of claim 18 further comprising a top layer disposed on a top side of said second insulative material.

20. The method of claim 1, wherein the device provides safe heat therapy to the subject while sleeping.
